(19) United States
(12) Patent Application Publication
(10) Pub. No.: US 2026/0035636 A1
(43) Pub. Date: Feb. 5, 2026

(54) METHOD FOR ENHANCING THE EFFICIENCY OF OIL EXTRACTION PROCESS

(71) Applicants: DSM IP Assets B.V., Maastricht (NL); Evonik Operations GmbH, ESSEN (DE)

(72) Inventors: Stephen Robert CHERINKO, Columbia, MD (US); Matthias DERNEDDE, Bruchkobel (DE); Michael DIEHL, Frankfurt (DE); Xiao Daniel DONG, Ellicott City, MD (US); Michael Benjamin JOHNSON, Baltimore, MD (US); Robert Cody KERTIS, Timmonsville, SC (US); Jochen LEBERT, Glattbach (DE); Neil Francis LEININGER, Winchester, KY (US); Kirt Lyvell MATTHEWS, SR., Fort Mill, SC (US); Holger PFEIFER, Hanau (DE); Horst PRIEFERT, Ostbevern (DE); Christian RABE, Grossostheim (DE); Shannon Elizabeth Ethier RESOP, Olney, MD (US); Joachim WINDAU, Warendorf (DE); Daniel VERKOEIJEN, Florence, SC (US); Gabriel ZAVODSKY, Brezova P. Bradlom (SK)

(21) Appl. No.: 19/329,364

(22) Filed: Sep. 15, 2025

Related U.S. Application Data
(63) Continuation of application No. 16/309,632, now abandoned.
(60) Provisional application No. 62/361,770, filed on Jul. 13, 2016.

(51) Int. Cl. C11B 1/10; A23K 10/12; A23K 20/158; C07C 51/48; C11B 1/02; C11B 1/14; C11B 3/16; C12N 1/06; C12P 7/6427; C12P 7/6432; C12P 7/6434; C12P 7/6472

(52) U.S. Cl. CPC C11B 1/10; C07C 51/48; C11B 1/02; C11B 1/025; C11B 1/14; C11B 3/16; C12N 1/06; C12P 7/6427; C12P 7/6432; C12P 7/6434; C12P 7/6472; A23K 10/12; A23K 20/158

(57) ABSTRACT

Disclosed herein are processes for obtaining a microbial oil comprising one or more polyunsaturated fatty acids (PUFAs) from one or more microbial cells by removing water from the cell fermentation broth or lysed cell composition before demulsification is conducted. Such process has the benefits of reduced demulsification time and reduced salt use. Further disclosed herein is microbial oil comprising one or more PUFAs that is recovered from microbial cells by the process described herein.

### CROSS REFERENCE TO RELATED APPLICATIONS

This application is a continuation of commonly owned copoending of U.S. application Ser. No. 16/309,632 filed Dec. 13, 2018 (now abandoned), which in turn is a US National Stage of International Application No. PCT/US2017/041686 filed Jul. 12, 2017, which claims the benefit of the filing date of U.S. Provisional Patent Application No. 62/361,770 filed Jul. 13, 2016, the entire contents of each of which are hereby incorporated by reference.

### BACKGROUND

It is desirable to increase the dietary intake of many beneficial nutrients. Particularly beneficial nutrients include fatty acids such as omega-3 and omega-6 long chain polyunsaturated fatty acids (LC-PUFAs) and esters thereof. Long chain omega-3 and omega-6 fatty acids are an essential part of the human diet that are currently derived mainly from fish oils or microbial oils.

Due to problems with overfishing, there is a need for an alternative sustainable source of omega-fatty acids such as eicosapentaenoic acid (EPA) and docosahexaenoic acid (DHA) that have demonstrated health benefits in humans. Such alternative source of omega-3 fatty acids are also needed for fish feed, due to the fact that farm-raised fish obtains its omega-3 fatty acids from supplement in the fish feed instead of from microalgae or marine phytoplankton in the wild.

Lipids for use in nutritional products and animal feed can be produced by microorganisms. Manufacturing a lipid in algae, for example, may include growing the algae and extracting the intracellular lipids from it. A good source for PUFA containing lipids are from oleaginous microorganisms such as algal strains of the order Thraustochytriales, algal strains of the genus Crypthecodinium, or fungal strain of the genus Mortierella, among many other microorganisms.

The industrial scale process for obtaining PUFA containing oil from microbial cells involves growing microorganisms that are capable of producing the desired oil in a fermentor or pond to produce a microbial cell biomass, and subsequently extracting the oil from the cell biomass. The process for extracting PUFA containing oil from microbial cells are costly, with some requiring energy intensive step such as heat to dry the cells, with some requiring organic solvent to recover PUFA oil, and yet some requiring chemicals and enzymes to break cells and emulsions. Heat can degrade and oxidize the PUFA containing oil and thus creates undesirable taste. Use of solvent requires costly equipment, high energy cost for solvent recovery, and implementation of waste treatment measures to reduce negative environmental impact. Use of the chemicals and enzymes increase the processing cost and also requires implementation of expensive waste disposal procedures. Furthermore, production in large scale requires equipment and containers to be suitably constructed to handle large volume. It creates yet another technical challenge and further increases processing costs.

Thus, it was the objective of the present invention to provide an effective method for extracting PUFA containing oils from microbial cells using less energy and materials and thus lower the overall cost of production. It was a further objective of the present application to provide a method for obtaining high quality PUFA containing oil.

### SUMMARY OF INVENTION

The present invention is directed to a method for enhancing demulsification of a fermentation broth containing lysed oleaginous microorganisms, comprising: a) removing water from the fermentation broth wherein the volume of the fermentation broth containing lysed oleaginous microorganisms is less than 60% of its original volume; and b) demulsify the fermentation broth by heating to a temperature of 60° C. to 110° C.

In some embodiments, the demulsification is enhanced by reducing the time of demulsification to at least ⅓ of the time that is needed for demulsification when step a) is not performed. In some embodiments, the method further comprises step c) recovering an oil from the fermentation broth.

In some embodiments, the recovering of oil is performed using an solventless extraction method.

In some embodiments, the amount of oil recovered is increased by at least 7% compared to the same method when step a) is not performed.

In some embodiments, the volume of the fermentation broth containing lysed oleaginous microorganisms in step a) is reduced to less than 70%, and preferably less than 80% of its original volume.

In some embodiments, removal of water in step a) is performed by heating the fermentation broth at a temperature not more than 110° C., preferably between 70° C. to 100° C., and more preferably between 80° C. and 90° C.

In some embodiments, step b) comprises adding an alkalizing agent, preferably caustic soda.

In some embodiments, pH of the fermentation broth in step (b) is adjusted to a pH value of 5.5 to 12, preferably 7.0 to 12.0, preferably 9.5 to 10.5, and more preferably 9.7 to 10.2.

In some embodiments, the temperature in step b) is between 85° C. and 95° C., and preferably about 90° C. In some embodiments, the method of any preceding claim, wherein the temperature in step b) is maintained for at least one hour, at least two hours, at least three hours, and at least four hours. In some embodiments, the temperature in step b) is maintained for between twenty four to seventy two hours, preferably twenty four to thirty six hours.

The present invention is also directed to a method for extracting a microbial oil comprising one or more polyunsaturated fatty acids from a fermentation broth containing oleaginous microorganisms, comprising: (a) lysing the oleaginous microorganisms in the fermentation broth to form a lysed cell composition; (b) removing water from the lysed cell composition wherein the volume of the lysed cell composition is reduced to less than 60% of its original volume; (c) heating up the lysed cell composition as obtained in step (b) to a temperature of 60° C. to 110° C.; and (d) recovering the microbial oil from the lysed cell composition.

In some embodiments, the volume of the lysed cell composition in step (b) is reduced to less than 70%, and preferably less than 80% of its original volume.

In some embodiments, removal of water in step (b) is performed by heating the fermentation broth at a temperature not more than 110° C., preferably between 70° C. to 100° C., and more preferably between 80° C. and 90° C.

In some embodiments, step (c) comprises adding an alkalizing agent, preferably caustic soda. In some embodiments, pH of the lysed cell composition in step (c) is adjusted to a pH value of 5.5 to 12, preferably 7.0 to 12.0, preferably 9.5 to 10.5, and more preferably 9.7 to 10.2.

In some embodiments, the temperature in step (c) is between 85° C. and 95° C., and preferably about 90° C.

In some embodiments, the temperature in step (c) is maintained for at least one hour, at least two hours, at least three hours, and at least four hours. In some embodiments, the temperature in step (c) is maintained for twenty four to seventy two hours, preferably twenty four to thirty six hours.

The present invention is also directed to a method for extracting a microbial oil comprising one or more polyunsaturated fatty acids from a fermentation broth containing oleaginous microorganisms, comprising: (a) removing water from the fermentation broth wherein the volume of the fermentation broth is reduced to less than 60% of its original volume; (b) lysing the oleaginous microorganisms in the fermentation broth to form a lysed cell composition; (c) heating up the lysed cell composition as obtained in step (b) to a temperature of 60° C. to 110° C.; and (d) recovering the microbial oil from the lysed cell composition.

In some embodiments, the volume of the fermentation broth in step (a) is reduced to less than 70%, and preferably less than 80% of its original volume.

In some embodiments, removal of water in step (a) is performed by heating the fermentation broth at a temperature not more than 110° C., preferably between 70° C. to 100° C., and more preferably between 80° C. and 90° C.

In some embodiments, step (c) comprises adding an alkalizing agent, preferably caustic soda. In some embodiments, pH of the lysed cell composition in step (c) is adjusted to a pH value of 5.5 to 12, preferably 7.0 to 12.0, preferably 9.5 to 10.5, and more preferably 9.7 to 10.2.

In some embodiments, the temperature in step (c) is between 85° C. and 95° C., and preferably about 90° C.

In some embodiments, the temperature in step (c) is maintained for at least one hour, at least two hours, at least three hours, and at least four hours. In some other embodiments, the temperature in step (c) is maintained for twenty four to seventy two hours, preferably twenty four to thirty six hours.

In any of the above described embodiments, the oleaginous microorganisms produce a microbial oil comprising one or more polyunsaturated fatty acids. In some embodiments, the polyunsaturated fatty acids comprise an omega-3 fatty acid, an omega-6 fatty acid, and mixtures thereof. In some embodiments, the polyunsaturated fatty acids comprise docosahexaenoic acid (DHA), eicosapentaenoic acid (EPA), docosapentaenoic acid (DPA), arachidonic acid (ARA), gamma-linolenic acid (GLA), dihomo-gamma-linolenic acid (DGLA), stearidonic acid (SDA), and mixtures thereof.

In some embodiments, the microbial cells are algae, yeast, fungi, protest, or bacteria cells. Such microbial cells may be from, for example, the genus Crypthecodinium, genus Mortierella, or order Thraustochytriales. In one embodiment, the microbial cells are from the order Thraustochytriales. In one embodiment, the microbial cells are from the genus Thraustochytrium, Schizochytrium, or mixtures thereof. In another embodiment, the microbial cells are from Mortierella alpina.

In the above embodiments, the lysed cell composition comprises liquid, cell debris, and microbial oil.

In some embodiments, the oil comprises at least 15% by weight eicosapentaenoic acid. In other embodiments, the oil comprises at least 30% by weight docosahexaenoic acid. In other embodiments, the oil comprises at least 30% by weight arachidonic acid.

The present invention is also directed to an oil obtained by the process described above. The present invention is also directed to a delipidated microbial biomass comprising less than 5% total polyunsaturated fatty acid.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 is a process flow diagram illustrating one embodiment of a solventless extraction method using a dewatering step immediately after the whole cell fermentation medium is pasteurized.

FIG. 2 is a process flow diagram illustrating one embodiment of a solventless extraction method using a dewatering step after the cells in whole cell fermentation medium is pasteurized and lysed.

FIG. 3 is the photograph of lysed cell composition treated by the dewatering step showing separation after 2 hours of coalescence treatment.

FIG. 4 is the photograph of lysed cell composition not treated by the dewatering step showing separation after 49 hours of coalescence treatment.

FIG. 5 shows phase composition during coalescing for an experiment with a dewatering step

FIG. 6 shows phase composition during coalescing for an experiment without a dewatering step.

### DETAILED DESCRIPTION

Embodiments identified herein as exemplary are intended to be illustrative and not limiting.

Fatty acids are classified based on the length and saturation characteristics of the carbon chain. Fatty acids present in a microbial oil can have from 4 to 28 carbon atoms and are termed short chain, medium chain, or long chain fatty acids based on the number of carbons present in the chain. Fatty acids are termed saturated fatty acids when no double bonds are present between the carbon atoms, and are termed unsaturated fatty acids when double bonds are present. Unsaturated long chain fatty acids are monounsaturated when only one double bond is present and are polyunsaturated when more than one double bond is present.

The microbial oil described herein refers to oil that comprises one or more PUFAs and is obtained from microbial cells.

Polyunsaturated fatty acids (PUFAs) are classified based on the position of the first double bond from the methyl end of the fatty acid; omega-3 (n-3) fatty acids contain a first double bond at the third carbon, while omega-6 (n-6) fatty acids contain a first double bond at the sixth carbon. For example, docosahexaenoic acid (DHA) is an omega-3 long chain polyunsaturated fatty acid (LC-PUFA) with a chain length of 22 carbons and 6 double bonds, often designated as “22:6n-3.” In one embodiment, the PUFA is selected from an omega-3 fatty acid, an omega-6 fatty acid, and mixtures thereof. In another embodiment, the PUFA is selected from LC-PUFAs. In a still further embodiment, the PUFA is selected from docosahexaenoic acid (DHA), eicosapentaenoic acid (EPA), docosapentaenoic acid (DPA), arachidonic acid (ARA), gamma-linolenic acid (GLA), dihomo-gamma-linolenic acid (DGLA), stearidonic acid (SDA), and mixtures thereof. In another embodiment, the PUFA is selected from DHA, EPA, and mixtures thereof. In another embodiment, the PUFA is selected from DHA, ARA, and mixtures thereof. In a further embodiment, the PUFA is DHA. In a further embodiment, the PUFA is EPA. In yet a further embodiment, the PUFA is ARA.

LC-PUFAs are fatty acids that contain at least 3 double bonds and have a chain length of 18 or more carbons or 20 or more carbons. LC-PUFAs of the omega-6 series include, but are not limited to, di-homo-gammalinoleic acid (C20:3n-6), arachidonic acid (C20:4n-6) (“ARA”), docosatetraenoic acid or adrenic acid (C22:4n-6), and docosapentaenoic acid (C22:5n-6) (“DPAn-6”). The LC-PUFAs of the omega-3 series include, but are not limited to, eicosatrienoic acid (C20:3n-3), eicosatetraenoic acid (C20:4n-3), eicosapentaenoic acid (C20:5n-3) (“EPA”), docosapentaenoic acid (C22:5n-3), and docosahexaenoic acid (C22:6n-3). The LC-PUFAs also include fatty acids with greater than 22 carbons and 4 or more double bonds including, but not limited to, C24:6(n-3) and C28:8(n-3).

The PUFAs can be in the form of a free fatty acid, salt, fatty acid ester (e.g. methyl or ethyl ester), monoacylglycerol (MAG), diacylglycerol (DAG), triacylglycerol (TAG), and/or phospholipid (PL).

Highly unsaturated fatty acids (HUFAs) are omega-3 and/or omega-6 polyunsaturated fatty acids that contain 4 or more unsaturated carbon-carbon bonds.

As used herein, a “lysed cell composition” refers to a composition comprising one or more lysed cells, including cell debris and other contents of the cell, in combination with microbial oil (from the lysed cells), and optionally, a fermentation broth that contains liquid (e.g., water), nutrients, and microbial cells. The terms “lyse” and “lysing” refer to a process whereby the wall and/or membrane of the microbial cell is ruptured. In one embodiment, the microbial cell is lysed by being subjected to at least one treatment selected from mechanical, chemical, enzymatic, physical, and combinations thereof. In another embodiment, the process comprises lysing the microbial cells comprising the microbial oil to form a lysed cell composition, wherein the lysing is selected from mechanical, chemical, enzymatic, physical, and combinations thereof.

As used herein, a “cell” refers to an oil-containing biomaterial, such as biomaterial derived from oleaginous microorganisms. Oil produced by a microorganism or obtained from a microbial cell is referred to as “microbial oil”. In one embodiment, microbial oil refers to a crude oil extracted from the biomass of the microorganism without further processing. Oil produced by algae and/or fungi is also referred to as algal and/or fungal oil, respectively.

As used herein, a “microbial cell” or “microorganism” refers to organisms such as algae, bacteria, fungi, yeast, protist, and combinations thereof, e.g., unicellular organisms. In some embodiments, a microbial cell is a eukaryotic cell. A microbial cell includes, but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect, schmuckeri, and Pythium, including but not limited to Pythium insidiosum.

In one embodiment, the microbial cells are from the genus Mortierella, genus Crypthecodinium, or order Thraustochytriales. In a still further embodiment, the microbial cells arc from Crypthecodinium cohnii. In yet an even further embodiment, the microbial cells are selected from Crypthecodinium cohnii, Mortierella alpina, genus Thraustochytrium, genus Schizochytrium, and mixtures thereof.

In a still further embodiment, the microbial cells include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia. In another embodiment, ARA is obtained from microbial cells from the genus Mortierella, which includes, but is not limited to, Mortierella elongata, Mortierella exigua, Mortierella hygrophila, Mortierella alpina, Mortierella schmuckeri, and Mortierella minutissima. In a further embodiment, ARA is obtained from microbial cells from Mortierella elongata IFO8570, Mortierella exigua IF08571, Mortierella hygrophila IF05941, Mortierella alpina IF08568, ATCC16266, ATCC32221, ATCC42430, CBS219.35, CBS224.37, CBS250.53, CBS343.66, CBS527.72, CBS529.72, CBS608.70, and CBS754.68, and mutants thereof. In a still further embodiment, the microbial cells are from Mortierella alpina.

In an even further embodiment, the microbial cells are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseum, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. In another embodiment, the microbial cells are from the order Thraustochytriales. In yet another embodiment, the microbial cells are from Thraustochytrium. In still a further embodiment, the microbial cells are from Schizochytrium. In a still further embodiment, the microbial cells are chosen from genus Thraustochytrium, Schizochytrium, or mixtures thereof.

The term “about” is intended to capture variations above and below the stated number that may achieve substantially the same results as the stated number.

The present invention provides methods and systems for enhancing demulsification of a fermentation broth containing lysed oleaginous microorganisms. The enhancement is achieved by dewatering the fermentation broth before extracting microbial oils from such oil containing microorganisms. The present invention also provides methods and systems for extracting microbial oil from oleaginous microorganisms contained in a fermentation broth by dewatering the fermentation broth before lysing cells in the broth. Dewatering of fermentation broth before subsequent oil extraction steps may have many advantages over the commonly used microbial oil solventless extraction methods, which do not include any dewatering step. For example, the method of invention is better than previous solventless extraction process as 1) far less or even no salt or enzyme is added during the demulsification step; 2) reduced time is taken at the demulsification step, 3) a better end product of biomeal is produced because such biomeal contains much less salt; and 4) equipment of far smaller volume can be used in downstream processing, such as a smaller centrifuge machine and smaller process container tank. In addition, reduced volume requires less time and energy to process the sample and thus saves on cost.

A typical process for obtaining microbial oil from oleaginous microorganisms involves growing microorganism that are capable of producing the desired oil in a fermentor or pond to produce a microbial cell biomass containing such oil; and subsequently extracting the oil from the biomass. One method for extracting oil involves organic solvent. It involves separating the biomass from the fermentation broth in which the biomass was grown; drying the microbial cell biomass followed by use of organic solvent such as hexane to extract the microbial oil, and subsequently removing the organic solvent by evaporation and thus leaving out the microbial oil. Alternatively, solventless extraction methods were used for extracting oil, in which no organic solvent was used. A typical solventless extraction method involves the following steps: pasteurizing or heating the cell-containing fermentation broth; lysing the cells to release microbial oil from the cells to form a lysed cell composition, which is in the form of a solution; treating the lysed cell composition with heat, salt, and pH adjustment in order to coalesce the oil droplets and remove emulsion from the solution. This is followed by further centrifuging the demulsified solution to separate oil from the rest of the solution.

In one embodiment of the present invention, a dewatering step is performed after both the pasteurization step and the cell lysis step which causes moisture level of the lysed cell composition to be reduced significantly. In another embodiment, the dewatering step is performed immediately after the pasteurization step and before the cell lysis step which causes the moisture level of the whole cell fermentation broth to be reduced significantly. In both embodiments, the volume of liquid composition to be processed is significantly reduced before the subsequent oil extraction steps and thus reduction of cost and increase of efficiency are achieved.

The choice of using one method over another depends on the physical property of the fermentation broth at the beginning of the solventless extraction process. If the viscosity of the fermentation broth at the beginning of the solventless extraction process is low, the additional step of dewatering may be performed right after the pasteurization step. If the viscosity of the fermentation broth at the beginning of the solventless extraction process is high, the additional step of dewatering may be performed after both the pasteurization step and the cell lysis step.

In some embodiments, the dewatering step comprises heating the whole cell fermentation broth or the lysed cell composition to at least 70° C., at least 75° C., at least 80° C., at least 85° C., at least 90° C., at least 95° C., at least 100° C., at least 105° C., or at least 110° C. In other embodiments, the dewatering step comprises heating the whole cell fermentation broth or the lysed cell composition to at between about 70° C. and about 110° C., at between about 70° C. and about 100° C., at between about 80° C. and about 100° C., or at between about 90° C. and about 100° C. In other embodiments, the dewatering step comprises heating the whole cell fermentation broth or the lysed cell composition to at about 85° C., at about 90° C., or at about 95° C.

In some embodiments, the temperature in the above dewatering step is maintained for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, or at least 30 hours.

In some embodiments, cells and/or a lysed cell composition can be heated in a system with an evaporator. In some embodiments, cells and/or a lysed cell composition can be heated in a system with an evaporator such that a portion of the water present in the cells and/or the lysed cell composition is removed by evaporation.

In some embodiments, the process comprises heating whole cell fermentation broth or lysed cell composition in a system with an evaporator to reduce the volume (or weight) of the whole cell fermentation broth or lysed cell composition to at least 30%, 35%, 40%, 45%, 50%, 55%, 60%, 65%, 70%, 75%, or 80% by volume (or weight) of the whole cell fermentation broth or lysed cell composition at the beginning of the dewatering step. In some embodiments, the process comprises heating whole cell fermentation broth or lysed cell composition in a system with an evaporator to reduce the volume (or weight) of the whole cell fermentation broth or lysed cell composition to 30% to 80%, 40% to 80%, 50% to 80%, 60% to 80%, 70% to 80%, 40% to 75%, 50% to 75%, 60% to 75%, 50% to 70%, or 55% to 65% by volume (or weight) of the whole cell fermentation broth or lysed cell composition at the beginning of the dewatering step.

In some embodiments, a lysed cell composition is in the form of an oil-in-water emulsion comprising a mixture of a continuous aqueous phase and a dispersed oil phase.

In some embodiments, lysing microbial cells results in the formation of an emulsion from endogenous materials in the cell or cell biomass including, but not limited to, proteins, phospholipids, carbohydrates, and combinations thereof. The terms “emulsion” and “emulsified” refer to a mixture of two or more immiscible phases or layers wherein one phase or layer is dispersed in another phase or layer. The terms “break,” “break-up,” “demulsify,” “demulsification,” “demulsifying,” and “breaking” refer to a process of separating immiscible phases or layers of an emulsion. For example, in some embodiments, a process of the present invention breaks an oil-containing emulsion from a single-phase to two or more phases. In some embodiments, the two phases include a light phase and a heavy phase. In some embodiments, a process of the present invention breaks an oil-containing emulsion into at least three phases. In some embodiments, the three phases are an oil phase, an emulsion phase, and an aqueous phase. In some embodiments, a process of the present invention breaks an oil-containing emulsion into at least four phases. In some embodiments, the four phases are an oil phase, an emulsion phase, an aqueous phase, and a solid phase.

The method further comprises heating the lysed and dewatered cell composition solution to break the emulsion. In some embodiments, the demulsification step comprises heating the lysed and dewatered cell composition solution to at least 60° C., at least 65° C., at least 70° C., at least 75° C., at least 80° C., at least 85° C., at least 90° C., at least 95° C., at least 100° C., at least 105° C., or at least 110° C. In other embodiments, the demulsification step comprises heating the cells or the lysed cell composition to between about 60° C. and about 110° C., between about 70° C. and about 100° C., between about 80° C. and about 100° C., or between about 90° C. and about 100° C. In other embodiments, the demulsification step comprises heating the cells or the lysed cell composition to about 85° C., at about 90° C., or at about 95° C.

As described above, in one embodiment, the dewatering step is performed after the pasteurization step, and thus effectively condensing the dissolved soluble solid components, such as salt, in a whole cell fermentation broth. The cells in the dewatered whole cell fermentation broth is then lysed to form a lysed cell composition. In another embodiment, the dewatering step is performed after the cell lysing step, and thus effectively condensing the dissolved soluble solid components, such as salt, from the lysed cell composition. The salt concentration in the lysed cell composition is increased after the dewatering step.

The method further comprises pasteurizing the cell fermentation broth before the dewatering step. In one embodiment, the pasteurization process comprising heating the cells at 60° C. for at least 1 hr, at least 1.5 hrs, or at least 2 hrs. In another embodiment, the pasteurization process comprising heating the cells at a temperature between 60-70° C. for at least 1 hr, at least 1.5 hrs, or at least 2 hrs. In another embodiment, the pasteurization process comprising heating the cells at (a temperature comprising) from 40° C. to (60° C. or) 70° C. in no more than 30 minutes or heating the cells at a rate of at least 0.5° C./minute. In one embodiment, the pasteurization process comprising using a pasteurization protocol such that the area under the temperature (° C.) versus time (minutes) graph is below 6,000° C.minute. In another embodiment, the pasteurization process comprising using a pasteurization protocol such that the area under the temperature (° C.) versus time (minutes) graph is below 13,000° C.minute. The area under the time versus temperature graph gives the amount of energy expended in heating the cells during the pasteurization process.

A particular advantage of the method of the present invention is that it can accelerate the demulsification step. In one embodiment, the time for conducting the demulsification process is reduced when the dewatering step is performed when compared to when the dewatering step is not performed. In another embodiment, the time to achieve the same demulsification effect is reduced to at least 60%, at least 45%, or at least 40% of the time required when compared to a process where the dewatering step is not performed. In another embodiment, the overall time for oil extraction is reduced when the dewatering step is performed compared to when the dewatering step is not performed. In another embodiment, the overall energy use for oil extraction is reduced when the dewatering step is performed compared to when the dewatering step is not performed.

In some embodiments, the temperature in the demulsification step is maintained for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, or at least 30 hours. In some embodiments, the temperature in the above demulsification step is maintained for between 10 and 36 hours, between 10 and 12 hours, between 10 and 14 hours, between 10 and 24 hours, between 12 and 36 hours, between 14 and 36 hours, between 16 and 36 hours, between 18 and 36 hours, between 20 and 36 hours, between 22 and 36 hours, between 24 and 36 hours, between 26 and 36 hours, between 28 and 36 hours, between 16 and 26 hours, between 18 and 26 hours, between 20 and 26 hours, between 22 and 26 hours, between 22 and 24 hours, between 23 and 25 hours, between 30 and 36 hours, or between 30 and 34 hours.

In some embodiments, the demulsification step further comprises pH adjustment. In some embodiments, the pH is adjusted to between 7 and 12, between 7.5 and 11.5, between 9.5 and 11.5, preferably between 10.0 and 11.0, and more preferably 10.3 to 10.7.

A further advantage of the method of the present invention is that it can reduce or eliminate the use of salt in breaking the emulsion. The method of the present invention further has the benefit of using little or no salt in lysing the cells. In the demulsification step of previous solventless extraction methods, salt is added to help breaking the emulsion. In addition, sometimes excessive amount of cell-wall breaking enzymes are added during the lysing step to help break the emulsion during and after the lysing step. As disclosed in the paragraphs above, the dewatering step allows the increase of concentration of salts in the whole cell fermentation broth or the lysed cell composition. This reduces the amount of salt needed for breaking the emulsion at the demulsification step or eliminates such need altogether. In one embodiment, less than 2% wt of salts, in particular sodium chloride, are used in the entire oil extraction process. In another embodiment, less than 1% wt of salts, in particular sodium chloride, are used in the entire oil extraction process. In another embodiment, less than 0.5% wt of salts, in particular sodium chloride, are used in the entire oil extraction process. In another embodiment, no salt is used in the entire oil extraction process. In one embodiment, less than 1% wt of cell-wall-breaking enzymes are used. In another embodiment, less than 0.5% wt of cell-wall-breaking enzymes are used. In another embodiment, less than 0.15% wt of cell-wall-breaking enzymes are used. In another embodiment, no cell-wall-breaking enzymes are used.

Yet another advantage of the method of the present invention is that it reduces the volume of the containers that is required in the oil extraction process. The reduced container volume carries the advantage of less equipment cost, less energy usage and higher mixing efficiency. In one embodiment of the invention, the container used during the demulsification step is reduced to at least 50%, at least 60%, or at least 70% of the container that is required if the dewatering step is not performed. Because of the reduction of container volume, the total agitation power can also be reduced. In another embodiment, the agitation power in the container used the demulsification step is reduced to at least 50%, at least 60%, or at least 70% of its original amount of power consumed if the dewatering step is not performed.

Another advantage of the method of the present invention helps with the demulsification step which results in a yield improvement and/or shorter demulsification time. Without being bound by theory, it is believed that in order for demulsification to occur, emulsified oil droplets need to coalesce into larger droplets. As the oil droplets become larger, it is easier to separate the oil from the water phase via centrifugation. By increasing the oil titer (L of oil/L of broth), the oil droplets are more concentrated in the broth and can more easily and effectively coalesce in order to form bigger droplets and ultimately be separated from the water phase via centrifugation. In addition to bringing the oil droplets closer together, it is believed that dewatering process also has the effect of increasing the salt concentration in the broth which helps to break the emulsion. In one embodiment, the amount of oil recovered using the above-mentioned dewatering process is increased by about 5-9% compared to the same method when the dewatering step is not performed. In one embodiment, the amount of oil recovered using the above-mentioned dewatering process is increased by at least 7% compared to the same method when the dewatering step is not performed. In another embodiment, the amount of oil recovered using the above-mentioned dewatering process is increased from about 85% to between 90-94%. In another embodiment, the amount of time for conducting the demulsification step has been reduced by about 12 hours. In another embodiment, the amount of time for conducting the demulsification step has been reduced from about 36 hours to about 24 hours.

Disclosed herein is a microbial oil or a biomeal obtained by any of the methods described herein.

Disclosed herein is a microbial oil that can be obtained from microbial cells by any of the processes disclosed herein. In some embodiments, the oil comprises at least 15% by weight eicosapentaenoic acid. In some embodiments, the oil comprises at least 30% by weight docosahexaenoic acid. In some embodiments, the oil comprises at least 30% by weight arachidonic acid.

In one embodiment, the microbial oil obtained and/or recovered by any of the processes described herein is a crude oil. In another embodiment, the oil described herein is a refined oil. A “crude oil” is an oil obtained from microbial cells without further processing. A “refined oil” is an oil obtained by treating a crude oil with standard processing of refining, bleaching, and/or deodorizing. Sec, e.g., U.S. Pat. No. 5,130,242. In some embodiments, refining includes, but is not limited to, base refining, degumming, acid treatment, alkali treatment, cooling, heating, bleaching, deodorizing, deacidification, and combinations thereof.

In some embodiments, the oil obtained using the method of the present invention comprises one or more PUFAs. In some embodiments, the oil comprises at least 10%, at least 15%, at least 20%, at least 25%, at least 30%, at least 35%, at least 40%, at least 45%, at least 50%, at least 60%, at least 70%, or at least 80% PUFA (by PUFA weight). In some embodiments, the oil comprises at least 10%, at least 15%, at least 20%, at least 25%, at least 30%, at least 35%, at least 40%, at least 45%, at least 50%, at least 60%, at least 70% or at least 80% DHA (by DHA weight), and/or at least 10%, at least 15%, or at least 20% DPA n-6 (by DPA n-6 weight), and/or at least 10%, at least 15%, at least 20% EPA, at least 25% EPA, at least 30% EPA, at least 35% EPA, at least 40% EPA, at least 45% EPA, or at least 50% EPA (by EPA weight), and/or at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% ARA (by ARA weight). In some embodiments, an oil comprises less than 50%, less than 40%, less than 30%, less than 20%, less than 15%, less than 10%, or less than 5% EPA (by EPA weight). In some embodiments, an oil comprises less than 50%, less than 40%, less than 30%, less than 20%, less than 15%, less than 10%, or less than 5% DHA (by DHA weight). In some embodiments, an oil comprises less than 10%, less than 5%, less than 2%, less than 1%, or less than 0.5% by weight of sterols.

In some embodiments, the above oil comprises at least 50%, at least 60%, at least 70%, at least 80%, at least 90%, at least 95%, or 50% to 95%, 50% to 90%, 50% to 85%, 50% to 80%, 50% to 75%, 60% to 95%, 60% to 90%, 60% to 85%, 70% to 95%, 70% to 90%, 70% to 85%, 75% to 95%, 75% to 90%, or 75% to 85%, by weight of triglycerides.

In some embodiments, the above triglycerides comprise at least 50%, at least 40%, at least 30%, at least 20%, at least 15%, at least 10%, or at least 5% by weight EPA. In some embodiments, the triglycerides comprise at least 10%, at least 20%, at least 30%, at least 35%, at least 40%, at least 50%, at least 60%, at least 70% or at least 80% by weight DHA. In some embodiments, the triglycerides comprise at least 10%, at least 20%, at least 30%, at least 35%, at least 40%, at least 45%, at least 50%, at least 55%, at least 60%, at least 65%, at least 70%, at least 75%, or at least 80% by weight ARA.

In some embodiments, the oil obtained using the method of the present invention comprises at least 40%, at least 50% or at least 60% by weight DHA, and/or less than 15%, less than 10%, or less than 8% by weight EPA. In come embodiments, the above oil comprises at least 70%, 80%, 90% or 95% by weight triglycerides. In one embodiment, the microbial oil is a crude oil. In another embodiment, the microbial oil is a refined oil.

In some embodiments, the oil obtained using the method of the present invention comprises at least 30%, at least 35% or at least 40% by weight DHA, and/or at least 10%, at least 15%, or at least 20% by weight EPA. In come embodiments, the above oil comprises at least 70%, 80%, 90% or 95% by weight triglycerides. In one embodiment, the microbial oil is a crude oil. In another embodiment, the microbial oil is a refined oil.

In some embodiments, the oil obtained using the method of the present invention comprises at least 40%, at least 45% or at least 50% by weight DHA, and/or less than 25%, less than 20%, or less than 15% by weight DPAn-6. In come embodiments, the above oil comprises at least 70%, 80%, 90% or 95% of by weight triglycerides. In one embodiment, the microbial oil is a crude oil. In another embodiment, the microbial oil is a refined oil.

In some embodiments, the oil obtained using the method of the present invention comprises at least 55%, at least 60% or at least 65% by weight DHA. In come embodiments, the above oil comprises at least 70%, 80%, 90% or 95% by weight triglycerides. In one embodiment, the microbial oil is a crude oil. In another embodiment, the microbial oil is a refined oil.

In some embodiments, the oil obtained using the method of the present invention comprises at least 30%, at least 35% or at least 40% by weight DHA, and/or less than 5%, less than 2%, or less than 1% by weight DPAn-6. In come embodiments, the above oil comprises at least 70%, 80%, 90% or 95% by weight triglycerides. In one embodiment, the microbial oil is a crude oil. In another embodiment, the microbial oil is a refined oil.

In some embodiments, the oil obtained using the method of the present invention comprises at least 25%, at least 30% or at least 35% by weight DHA, and/or at least 10%, at least 15%, or at least 20% by weight EPA, and/or less than 10%, less than 5%, or less than 3% by weight DPAn-6, and/or less than 15%, less than 10%, or less than 7% by weight DPAn-3. In come embodiments, the above oil comprises at least 70%, 80%, 90% or 95% by weight triglycerides. In one embodiment, the microbial oil is a crude oil. In another embodiment, the microbial oil is a refined oil.

In some embodiments, the oil obtained using the method of the present invention comprises at least 40%, at least 45%, or at least 50% by weight ARA. In come embodiments, the above oil comprises at least 70%, 80%, 90% or 95% by weight. In one embodiment, the microbial oil is a crude oil. In another embodiment, the microbial oil is a refined oil.

The methods of the present invention allow a very effective extraction of oil from biomass. By using the methods of the present invention, it is possible to remove more oil from the biomass and thus far less oil remains in the delipidated biomass. Thus, in one embodiment, the present invention relates to a delipidated biomass comprising less than 10% total fatty acid. In another embodiment, the present invention relates to a delipidated biomass comprising less than 5% total fatty acid.

Effective culture conditions for a microbial cell for use with the invention include, but are not limited to, effective media, bioreactor, temperature, pH, and oxygen conditions that permit oil production. An effective medium refers to any medium in which a microbial cell, e.g., Thraustochytriales microbial cell, is typically cultured. Such media typically comprises an aqueous medium having assimilable carbon, nitrogen, and phosphate sources, as well as appropriate salts, minerals, metals, and other nutrients, such as vitamins. Microbial cells for use with the present invention can be cultured in conventional fermentation bioreactors, shake flasks, test tubes, microtiter dishes, and petri plates.

In some embodiments, an oil obtained according to any of the processes described herein, the delipidated biomass, or combinations thereof can be used directly as a food or food ingredient, feed or feed supplement for any non-human animal (e.g., those whose products (e.g., meat, milk, or eggs) are consumed by humans); and food supplements. The term “animal” refers to any organism belonging to the kingdom Animalia and includes any human animal, and non-human animal from which products (e.g., milk, eggs, poultry meat, beef, pork, lamb, and fish meat) are derived. In some embodiments, the oil and/or biomass can be used in feeding sea animals considered as seafood. Seafood is derived from, without limitation, fish, shrimp and shellfish. The term “products” includes any product derived from such animals, including, without limitation, meat, eggs, milk or other products. When the oil and/or biomass is fed to such animals, polyunsaturated oils can be incorporated into the flesh, milk, eggs or other products of such animals to increase their content of these oils.

### EXAMPLES

### Example 1

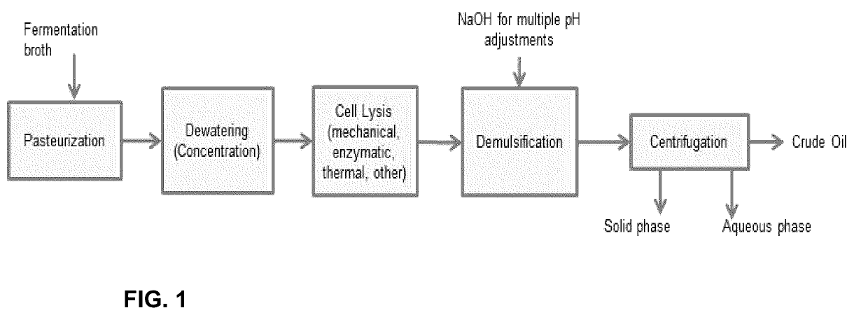

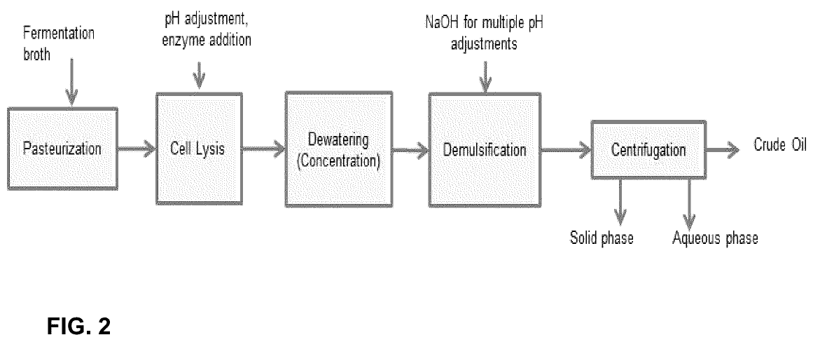

As depicted in FIGS. 1 and 2, the microbial cell suspension can be dewatered both before, during, or after the lysis of the microbial cells. One particular example of dewatering after cell lysis is explained below.

An unwashed cell broth (141.8 kg) containing microbial cells (Schizochytrium sp.) was pasteurized at 60° C. for 1 hour. After pasteurization the pH was 7.4, and the total solid content was 16.7%. The broth was divided equally and transferred into two 100 liter agitated tanks. While controlling the temperature at 60° C., Alcalase® enzyme (available from Novozymes (Franklinton, NC)) was added in an amount of 0.15% based on weight of the cell broth. The broth was held for 2 hours with the agitation speed at 200 RPM, and pH controlled at 7.5 with 20% NaOH solution. After that, the broth temperature was increased to 90° C. with all the head space ports open for broth evaporation. About 13 hours later, the broth in the two tanks were combined and the evaporation process was continued for another 8 hours until the total solid content in the broth reached 36.5%. The total evaporation time was 21 hours. The volume reduction of the fermentation broth was 54.4%.

In the next step, the demulsification process was performed. The pH was adjusted from 5.8 to 10.5 using 20% NaOH solution. 7.6 kg of NaOH solution was used. The broth was held at 90° C. with an agitation speed of 200 rpm and all ports closed except a small vapor vent line. 8 hours later, the pH dropped to 9.5, and 0.77 kg of 20% NaOH solution was added to bring pH up to 10.0. At about 26 hours later, pH was adjusted to 7.6 with 3.9 kg of 3NH₂SO₄. The temperature was reduced to 80° C. The above demulsification process produces phase separation of an oil phase, an emulsion phase, and an aqueous phase.

Next, oil was separated from the lysed cell composition by centrifugation (Alfa Laval Disc Stack Centrifuge, LAPX 404/Clara 20). The extraction yield was 91.61%. Comparing to a previous experiment without the dewatering step, the amount of time for conducting the demulsification step has been reduced ½ or 24 hours.

### Example 2

An unwashed cell broth (157.4 kg) containing microbial cells (Schizochytrium sp.) was pasteurized at 60° C. for 1 hour. Then pH of the broth was adjusted to 7.5 and Alcalase® enzyme (available from Novozymes (Franklinton, NC)) was added in an amount of 0.15% based on the weight of the cell broth. The broth was agitated at a speed of 140 RPM, and the temperature was maintained at 60° C. for 2 hours. After 2 hours, the lysed cell composition was heated to 90° C. and allowed to evaporate from an initial total solid content of 16.9% to a final total solid content of 30.5%. This resulted in 87.2 kg of concentrated broth containing microbial oil and cell debris. The volume reduction was 44.5%. The pH of the lysed and concentrated cell composition was adjusted to 10.5 by adding 2.6 kg of 50% NaOH. The broth was agitated at 140 RPM, and held for 24 hours. During the holding period, there was one additional pH adjustment with NaOH to bring the pH back to 10 when the pH had fallen below 9. At the end of the coalescence period, the pH was adjusted from 9.7 to 8.0 with 2.8 kg of 3NH₂SO₄ and the temperature was lowered to 80° C. The crude oil phase that had formed was separated from the lysed cell composition by centrifuging (Alfa Laval Disc Stack Centrifuge, LAPX 404/Clara 20). The extraction yield was 91.8%.

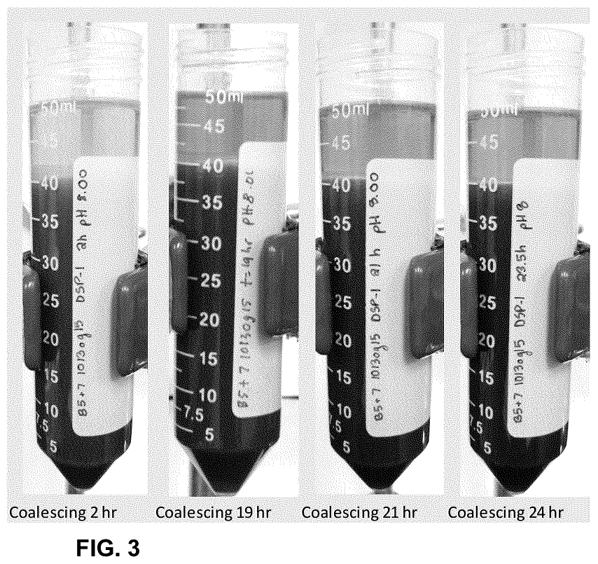

As evidenced by the time trend shown in FIG. 3, the fermentation broth processed by including the dewatering step showed good oil separation in as little as 2 hours after coalescence treatment.

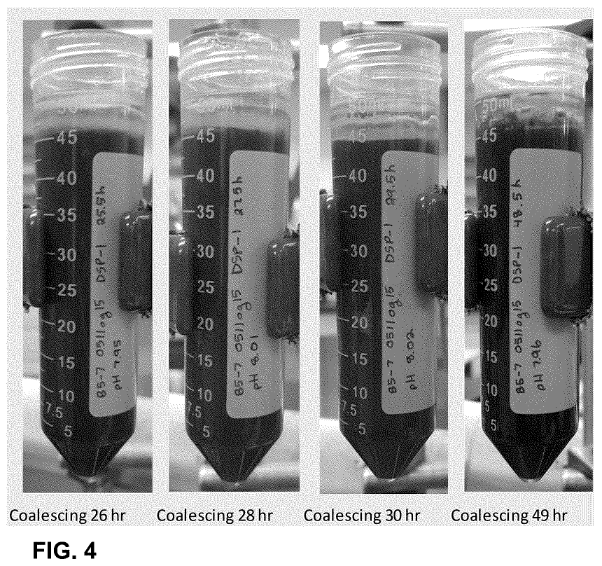

When compared to a control experiment that did not dewater the lysed cell composition (see FIG. 4), it has been shown that the emulsion phase lasted for a longer period of time before it could be separated from the heavy phase. Some of the emulsion was mixed with free oil and ended into the centrifuge light phase, which resulted in oil with high moisture content and required a further refining step.

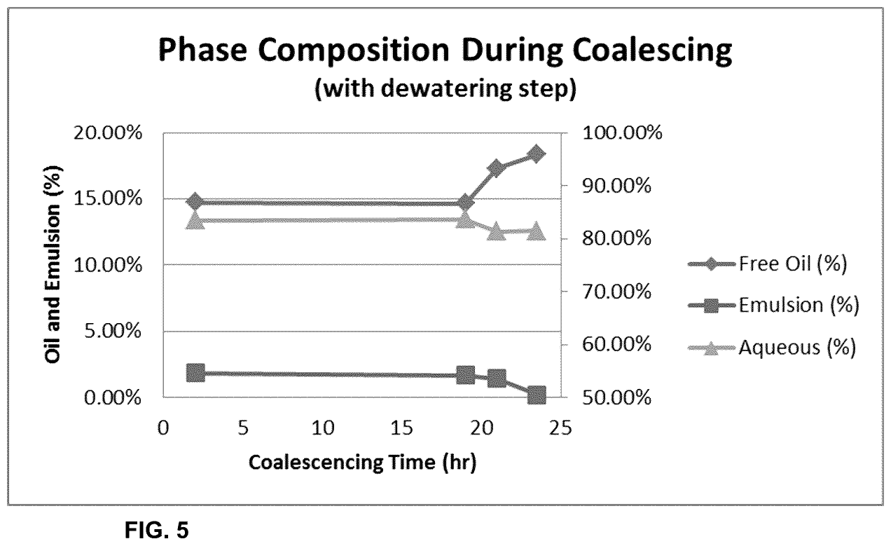

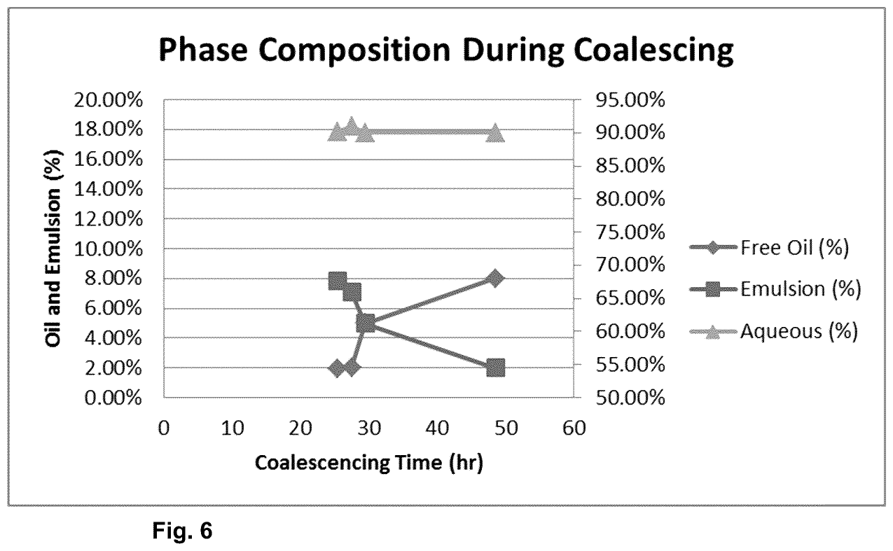

The volume of free oil phase, emulsion phase, and the aqueous phase was estimated, and the percentage of each phase over the total volume was calculated to show the progression of oil coalescing. Comparing FIG. 5 to FIG. 6, it clearly demonstrated the benefit of dewatering step. Without a dewatering step, free oil phase was only 2% of the total volume at 26 hrs (FIG. 6), while with a dewatering step, free oil phase was already 15% of total volume at 2 hrs (FIG. 5). In the experiment with the dewatering step, due to the water reduction, the oil concentration was about doubled, and the volume of oil phase was 18% of the total volume at the end of coalescing, while for the experiment without the dewatering step, the volume of free oil phase was only 8% of the total volume at the end.

What is claimed is:

1. A method for enhancing demulsification of a fermentation broth containing lysed oleaginous microorganisms, comprising:
a) removing water from the fermentation broth wherein the volume of the fermentation broth containing lysed oleaginous microorganisms is less than 60% of its original volume;
b) demulsify the fermentation broth by heating to a temperature of 60° C. to 110° C.; and
c) recovering an oil from the fermentation broth,
wherein the demulsification is enhanced by reducing time of demulsification to at least ⅓ of the time that is needed for demulsification when step a) is not performed, and wherein less than 0.15% wt of salts or no salt are used during the entire oil extraction process and wherein the recovering of oil is performed without use of a solvent.

2. The method of claim 1, wherein the amount of oil recovered is increased by at least 7% compared to the same method when step a) is not performed.

3. The method of claim 1, wherein the volume of the fermentation broth containing lysed oleaginous microorganisms in step a) is reduced to less than 70.

4. The method of claim 1, wherein removal of water in step a) is performed by heating the fermentation broth at a temperature between 70° C. to 100° C.

5. The method of claim 1, wherein step b) comprises adding an alkalizing agent.

6. The method of claim 5, wherein pH of the fermentation broth is adjusted to a pH value of 5.5 to 12.

7. The method of claim 1, wherein the temperature in step b) is between 85° C. and 95° C.

8. The method of claim 1, wherein the temperature in step b) is maintained for at least one hour.

9. The method of claim 5, wherein the temperature in step b) is maintained for twenty four to seventy two hours.

10. A method for extracting a microbial oil comprising one or more polyunsaturated fatty acids from a fermentation broth containing oleaginous microorganisms, comprising:
(a) lysing the oleaginous microorganisms in the fermentation broth to form a lysed cell composition;
(b) removing water from the lysed cell composition wherein the volume of the lysed cell composition is reduced to less than 60% of its original volume;
(c) heating up the lysed cell composition as obtained in step (b) to a temperature of 60° C. to 110° C.; and
(d) recovering the microbial oil from the lysed cell composition.

11. The method of claim 10, wherein the volume of the lysed cell composition in step (b) is reduced to less than 70%, and preferably less than 80% of its original volume.

12. The method of claim 11, wherein removal of water in step (b) is performed by heating the lysed cell composition at a temperature not more than 110° C., preferably between 70° C. to 100° C., and more preferably between 80° C. and 90° C.

13. The method of claim 10, wherein step (c) comprises adding an alkalizing agent, preferably caustic soda.

14. The method of claim 10, wherein pH of the lysed cell composition is adjusted to a pH value of 5.5 to 12, preferably 7.0 to 12.0, preferably 9.5 to 10.5, and more preferably 9.7 to 10.2.

15. The method of claim 10, wherein the temperature in step (c) is between 85° C. and 95° C., and preferably about 90° C.

16. The method of claim 10, wherein the temperature in step (c) is maintained for at least one hour, at least two hours, at least three hours, and at least four hours.

17. The method of claim 16, wherein the temperature in step (c) is maintained for twenty four to seventy two hours, preferably twenty four to thirty six hours.

18. A method for extracting a microbial oil comprising one or more polyunsaturated fatty acids from a fermentation broth containing oleaginous microorganisms, comprising:
(a) removing water from the fermentation broth wherein the volume of the fermentation broth is reduced to less than 60% of its original volume;
(b) lysing the oleaginous microorganisms in the fermentation broth to form a lysed cell composition;
(c) heating up the lysed cell composition as obtained in step (b) to a temperature of 60° C. to 110° C.; and
(d) recovering the microbial oil from the lysed cell composition.

19. The method of claim 18, wherein the volume of the fermentation broth in step (a) is reduced to less than 70%, and preferably less than 80% of its original volume.

20. The method of claim 18, wherein the oleaginous microorganisms produce a microbial oil comprising one or more polyunsaturated fatty acids.

21. An oil obtained by the process of claim 18.

22. A delipidated microbial biomass comprising less than 5% total polyunsaturated fatty acids.

23. A method for enhancing demulsification of a fermentation broth containing lysed oleaginous microorganisms, comprising:
a) removing water from an original volume of the fermentation broth to reduce the original volume of the fermentation broth containing lysed oleaginous microorganisms in an amount of 60% to 80% to thereby obtain a reduced volume of dewatered fermentation broth containing lysed oleaginous microorganisms;
b) demulsifying the dewatered fermentation broth containing lysed oleaginous microorganisms by heating the dewatered fermentation broth to a temperature of 60° C. to 110° C. so as to obtain a demulsified fermentation broth in a time that is reduced by at least 12 hours as compared to the time needed for demulsifying the fermentation broth if step a) is not performed; and
c) conducting a solventless extraction of oil from the demulsified fermentation broth to thereby recover the oil therefrom; wherein
less than 0.15 wt. % of salts or no salts are used during oil extraction processing according to steps a) and b).
